(19) United States
(12) Patent Application Publication
(10) Pub. No.: US 2009/0087462 A1
(43) Pub. Date: Apr. 2, 2009

(54) Use of pyrimidine derivatives for cosmetic purposes

(76) Inventors: Marcella Trembley, Basel (DE); Juergen H. Vollhardt, Ramlinsburg (CH)

Correspondence Address: NIXON & VANDERHYE, PC, 901 NORTH GLEBE ROAD, 11TH FLOOR, ARLINGTON, VA, 22203, US

(21) Appl. No.: 11/921,656

(22) PCT Filed: Jun. 12, 2006
(86) PCT No.: PCT/EP2006/005609

§ 371 Date: Dec. 6, 2007

(51) Int. Cl. A61K 8/49; C07D 487/04; A61K 9/10; A61K 31/519

(52) U.S. Cl. 424/401; 544/280; 514/252.16; 424/59; 424/484

(57) ABSTRACT

The present invention is directed to the use of certain pyrimidine derivatives for cosmetic purposes, i.e. for the preparation of a composition for providing a cosmetic effect. It was found that said pyrimidine derivatives are particularly useful for treating wrinkles but also for thickening the epidermis, for improving hair growth and for treating grey hair. The present invention is also directed to a composition containing said pyrimidine derivatives. In particular, said composition is a cosmetic preparation.

The present invention is directed to the use of certain pyrimidine derivatives for cosmetic purposes, i.e. for the preparation of a composition for providing a cosmetic effect. It was found that said pyrimidine derivatives are particularly useful for treating wrinkles but also for thickening the epidermis and for improving hair growth. The present invention also relates to a composition containing said pyrimidine derivatives and a further active ingredient. In particular, said composition is a cosmetic preparation.

It is known that 2-piperazinopyrimidine derivatives exhibit bioactive properties. In this regard it can be referred to e.g. EP-A 192 783.

U.S. Pat. No. 4,959,368 discloses a pharmaceutical composition comprising certain 2-piperazinopyrimidine derivatives or a pharmaceutically acceptable salt thereof. The compounds are said to have activities of promoting growth of nerve cells, forming neurites, and restoring motor functions, and are said to be useful as an agent for treating diseases of peripheral and central nerves. However, the document is silent on any cosmetic effects of the pyrimidine derivatives disclosed therein.

It is known from EP-A 743 066 that pyrimidine derivatives, which are structurally related to the compounds disclosed in EP-A 192 783 and U.S. Pat. No. 4,959,368, are useful as wound-healing agents. The document discloses a wound-healing agent comprising as an active ingredient a 2-substituted-6-alkyl-5-oxo-5,6-dihydro(7H)pyrro[3,4-d]pyrimidine compound or a 2-substituted-7-alkyl-6-oxo-5,6-dihydro(7H)pyrro[2,3-d]pyrimidine compound. The wound-healing agent is said to be useful for healing of wounds, for example, wounds by injuries, wounds by various accidents or disasters, burns, scalds, bone fractures, tooth extraction wounds, operative wounds caused at affected sites or peripheries thereof during surgical operations, body epithelial or endothelial ulcers, wounds such as keloids, texture injuries of gastrointestinal mucosae, hepatic injuries, bone damages, pseudoarthroses, necrosis of femoral head, ligamenteous damages, periodontal damages, vascular damages, myocardial infarction, arterial sclerosis, post-PTCA re-perfusion disorders, injuries by drugs or radiations, decubiti by some causes, and hemorrhoids. However, the document is silent on any cosmetic effects of the pyrimidine derivatives disclosed therein.

WO 03/060082 discloses that the pyrimidine derivatives known from EP-A 743 066 are also useful to promote stem cell migration and proliferation. The document discloses reagents and methods for proliferating stem cells in an animal in need thereof and producing stem cells that can be reintroduced into an animal in need thereof to alleviate neurological disorders. The stem cells which may be in situ stimulated and proliferated may be epidermal stem cells. However, cosmetic preparations are not disclosed.

Human skin consists of several compartments, e.g. epidermis, dermis, follicles and subdermis. Specific cell types and their activities contribute to the specific tasks of each compartment. Cells can be found in various stages of differentiation, particularly in the epidermis and dermis. The more pronounced the degree of differentiation, the more specialized and dedicated a cell is. It is now known for a couple of years that human skin also contains epidermal stem cells which are multipotent and thus have various options to differentiate through various stages into more specific cells. Epidermal stem cells provide the basis for basic epidermal renewal and are involved in hair growth, too. If there is a careful balance between providing newly differentiated and specialized cells from stem cells or progenitor cells and loss through differentiation due to other intrinsic or extrinsic factors skin and hair keeps its healthy status. Casual factors or external factors such as a raw climate, wind, irritation by the sun, rain and snow, however, disturb this normal condition of the skin, and in particular the barrier function of the skin. As a net result there appears roughness, formation of scales (for example on the scalp), an excessive keratinization and similar phenomena. Furthermore, in the course of aging of the skin various signs appear that are especially manifested by a change in the structure and function of the skin. One of these signs is the appearance of fine lines and deep wrinkles, the size and number of which increases with age. The microrelief of the skin becomes less uniform and is of unisotropic nature. In parallel with age the skin becomes more sensitive towards disturbing influences, either intrinsic or extrinsic, which may result in itch, redness or even darkened spots, in particular on hands and the facial area due to pigmentation disorders. These unwanted signs may lead to an undesired age judgment of a person. Cosmetic preparations are essential for skin care. One aim of skin care in the cosmetic sense is to strengthen or rebuild the skin's natural function as a barrier against environmental influences (e.g. dirt, chemicals, microorganisms) and against the loss of endogenous substances (e.g. water, natural fats, electrolytes). If this function becomes impaired, increased resorption of toxic or allergenic substances or attack by microorganisms may result, leading to toxic or allergic skin reactions.

Another aim of skin care is to compensate for the loss by the skin of lipids and water caused by daily washing. This is particularly important when the natural regeneration ability is inadequate. Furthermore, skin care products should protect against environmental influences, in particular against sun and wind, and delay skin aging.

Another aim of skin care is not even only to prevent or to delay skin aging but also to restore functional losses of the skin due to aging effects. Such effects include thinning of the epidermis and decreased waviness of the basal layer, barrier impairment, sensitive skin, decrease of anti-oxidant potential, increased MMP activity, itchiness, likeliness of chronic inflammation, male baldness, hair greying. In general aging results also in changes in the regulation of biochemical processes, impaired recruitment of cells and slowing down in cell renewal.

Strengthening or thickening of the epidermis can rebuild the skin's barrier ability and is therefore of significant cosmetic value. It also is known that this is a major strategy to fight further extrinsic causes of aging.

Of particular importance for anti-aging cosmetics is to inhibit the senescence of skin cells in order to keep their regular metabolic level on a constant and beneficial level.

Hair loss or alopecia is a common affliction of humans. The most common form of hair loss in both males and females is patterned baldness or androgenic alopecia. Hair follicles range in size from small, superficial, vellus follicles to large, deep, terminal follicles. The cyclic growth phases of hair follicles are telogen (resting), anagen I-III (developing), anagen IV-VI (growing) and catagen (involuting).

In the development of androgenic alopecia there is the gradual diminution of follicle size, with conversion of large, terminal follicles, producing thick, pigmented hair fibers (terminal hair) to small vellus follicles producing fine, non-pigmented hair fibers (vellus hair). In addition, the proportion of growing anagen follicles declines. Hair growth happens within the hair follicle and is controlled by the papilla region as a key signaling center. Even the precise molecular basis of male pattern baldness is still unknown although a hereditary factor is thought to play a role (Nyholt et. al. J. Invest Dermatol. 121 1561-1564, 2003) and there is also most likely a disturbance in the orchestrating events within the hair follicle.

Another sign of aging manifests itself in “grey hair”. The whitening of hair is called canities. It happens due to no further inclusion of pigments at the instance of its conception in the papilla. Even when the papilla producing a white hair contains melanocytes, they are incapable of producing coloured pigments. It has also been discovered that melanocytes were not only to be found at the bottom of the dermal papilla, but also in a reservoir situated higher in the external epithelial sheath. These melanocytes are dormant and produce no pigments. Some of these are recruited by the hair follicle to repopulate its lower part when it begins to regenerate itself at the end of the telogen phase. Once they have been selected, in healthy hair these melanocytes are reactivated and the production of melanin begins once more. Surprisingly the reservoir is still to be found in white hair follicles. However, some sort of recruitment deficiency or pigmentation deficiency obviously leads to canities.

It is one purpose of this invention to provide a technology to remove such orchestrating and recruitment disturbances in order to regain full thickness and coloured hair.

A wide variety of literature exists regarding cosmetic preparations, in particular for cosmetic preparations for treating wrinkles and for promoting hair growth. For examples of the extensive literature see e.g. GB 906 000, EP-A 699 429 or WO 03/086342.

The degree of the skin conditions mentioned above may vary inter-individually. In severe cases cosmetic preparations may not provide a sufficient alleviating effect and hence only a medical treatment of the corresponding skin disorders is appropriate. Accordingly, pharmaceutical preparations are useful for the treatment of said skin disorders, in particular for the treatment and/or the prevention of a disorder selected from the group consisting of wrinkles, acne, photo-damage, oxidative stress phenomena, cellulite, pigmentation disorders, thinned epidermis, hair loss and senescence of skin cells.

The problem to be solved by the present invention is the provision of compounds which are useful for preventing and/or treating wrinkles, thickening of the epidermis and preventing and/or treating hair loss and canities but which are also useful in the prevention and/or treatment of other conditions observed with skin aging due to environmental or other external influences or due to intrinsic ageing. The compounds should have an activity which is comparable to the activity of known compounds but preferably is better than the activity of the prior art compounds.

This problem is solved on the basis of the unexpected finding that certain pyrimidine derivatives of the prior art are useful for cosmetic purposes, preferably for preventing and/or treating wrinkles and hair loss and thickening the epidermis, but also for ameliorating the effects of aging of the skin, which may be caused by external or environmental hazards or by the natural aging of the skin.

Accordingly, the present invention provides the use of a pyrimidine derivative represented by general formula (I)

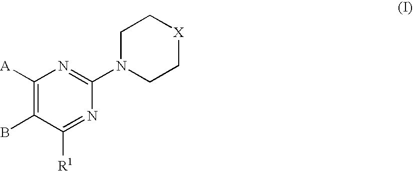

wherein
R¹ is H, C₁-C₁₂-alkyl, C₁-C₆-oxyalkyl, C₁-C₆-thioalkyl, C₁-C₆-alkylene-C₁-C₆-oxyalkyl, C₁-C₆-alkylene-CONH₂, CO—C₁-C₆-alkyl, or C₁-C₆-alkylene-OCO—C₁-C₆-alkyl;
X is —O—, —S—, —CH—, >CH—C₁-C₆-alkyl or >NR² wherein R² is —H, OH, C₁-C₆-alkyl, C₂-C₆-alkenyl, phenyl, benzyl, CH(phenyl)₂, CO—C₁-C₂₀-alkyl, CO₂—C₁-C₂₀-alkyl, or SOₙ—C₁-C₂₀-alkyl or SOₙ—C₆-C₇-aryl (wherein index n is an integer of 0 to 2);
A is H, NH₂, NH—C₁-C₆-alkyl, C₁-C₆-oxyalkyl, or CO₂—C₁-C₆-alkyl;
B is H, CO₂—C₁-C₆-alkyl, CON(C₁-C₁₂-alkyl)₂, C₁-C₆-oxyalkyl, or CH₂CH₂OH; or
A and B together with the carbon atoms to which they are attached form a 5 to 7-membered carbocyclic ring or a heterocyclic ring having N, O or S as the hetero atom; or a cosmetically acceptable salt thereof;
   for the preparation of a topical composition for providing a cosmetic effect.

Preferably, X is —O—, —CH₂—, >CH—C₁-C₆-alkyl or >NR². In case that A and B together with the carbon atoms to which they are attached form a 5 to 7-membered carbocyclic ring or a heterocyclic ring having N, O or S as the hetero atom, A and B preferably comprise less than 10 carbon atoms, more preferably less than 7 carbon atoms, most preferably less than 5 carbon atoms.

In a preferred embodiment of the present invention the pyrimidine derivative is represented by general formula (II)

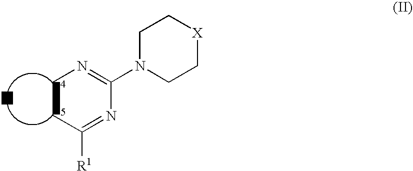

wherein R¹ and X are defined as above and wherein
Y is a moiety selected from the group consisting of the moieties represented by general formulae (III-A) to (III-J)

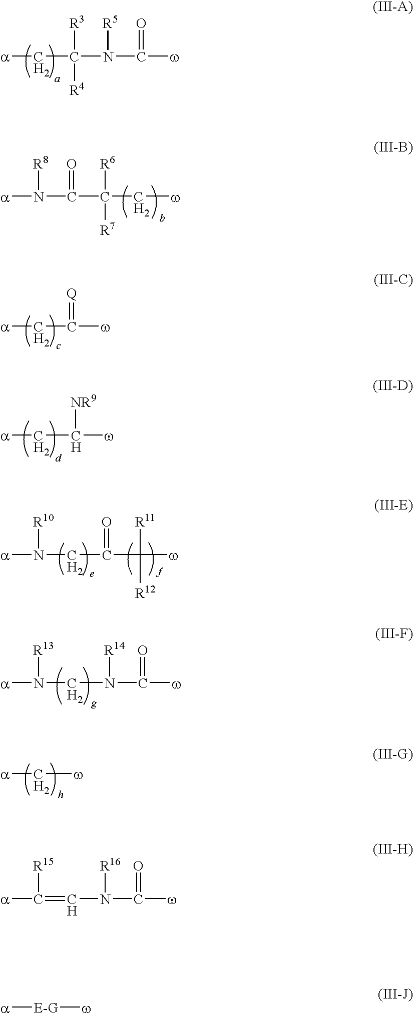

wherein
α and ω are the positions to be bound to positions 4 and 5 of the pyrimidine ring in general formula (II);
indices a and b each are independently an integer of 0 to 2;
indices c and d each are independently an integer of 2 to 4;
indices e and f each are independently an integer of 0 to 2, provided that 1≦(e+f)≦3;
index g is 1 or 2;
index h is 3 or 4;
Q is ═O or ═NR² (wherein R² is independently defined as above); E-G is —OCH₂CH₂—, —OC(CH₃)═CH—, —CH₂OCO—, —OCOCH₂—, —CH₂CH(CH₃)OCO—, —N(CH₃)CH₂CHr, —CH═CH—CH═CH—, —CH═C(OCH₃)═CH—, or

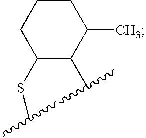

R³, R⁴, R⁶ and R⁷ each are independently H, C₁-C₁₂-alkyl, C₁-C₆-oxyalkyl, C₁-C₆-alkylene-C₁-C₆-oxyalkyl, C₁-C₆-alkylene-CONH₂, CO—C₁-C₆-alkyl, or C₁-C₆-alkylene-OCO—C₁-C₆-alkyl; and
R⁵, R⁸, R⁹, R¹⁰, R¹¹, R¹², R¹³, R¹⁴, R¹⁵ and R¹⁶ each are independently H or C₁-C₆-alkyl; or a cosmetically acceptable salt thereof.

Preferably, position α in general formulae (III-A) to (III-J) is bound to position 4 of the pyrimidine ring in general formula (II) and position ω in general formulae (III-A) to (III-J) is bound to position 5 of the pyrimidine ring in general formula (II).

Preferably, Y is a moiety selected from the group consisting of the moieties represented by general formulae (III-A) to (III-F), more preferably consisting of the moieties represented by general formulae (III-A) and (III-B). Preferably, Q is ═O. Preferably, X is —O—, —CH₂—, >CH—C₁-C₆-alkyl or >NR².

In a preferred embodiment of the present invention the pyrimidine derivative is a 5-oxo-5,6-dihydro(7H)pyrro-[3,4-d]pyrimidine derivative represented by general formula (IV-A) or a 6-oxo-5,6-dihydro(7H)pyrro-[2,3-d]pyrimidine derivative represented by general formula (IV-B)

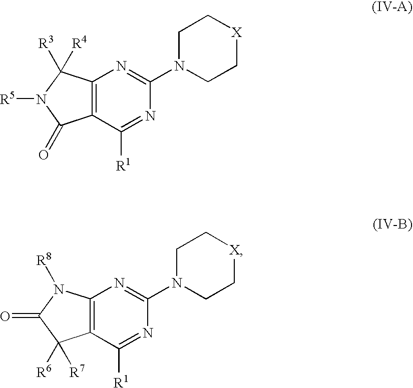

wherein X, R¹, R³, R⁴, R⁵, R⁶, R⁷ and R⁸ are defined as above.

For the purpose of the present specification the terms “alkyl”, “alkylene”, “alkeny”, “oxyalkyl” and “thioalkyl” denote a linear, cyclic and/or branched alkyl, alkylene, alkenyl, oxyalkyl and thioalkyl moiety, respectively.

Preferably, the moieties C₁-C₈-alkyl and C₁-C₁₂-alkyl are selected from the group consisting of CH₃, CH₂CH₃, CH₂CH₂CH₃, CH(CH₃)₂, CH₂CH₂CH₂CH₃, CH(CH₃)CH₂CH₃, CH₂CH(CH₃)₂, C(CH₃)₃, and CH₂CH₂CH₂CH₂CH₂CH₃.

Preferably, the moiety C₁-C₆-oxyalkyl is selected from the group consisting of OCH₃, OCH₂CH₃, OCH₂CH₂CH₃, OCH(CH₃)₂, OCH₂CH₁₂CH₂CH₃, OCH(CH₃)CH₂CH₃, OCH₂CH—(CH₃)₂, OC(CH₃)₃, and OCH₂CH₂CH₂CH₂CH₂CH₃.

Preferably, the moiety C₁-C₆-thioalkyl is selected from the group consisting of SCH₃, SCH₂CH₃, SCH₂CH₂CH₃, SCH(CH₃)₂, SCH₂CH₂CH₂CH₃, SCH(CH₃)CH₂CH₃, SCH₂CH—(CH₃)₂, SC(CH₃)₃, and SCH₂CH₂CH₂CH₂CH₂CH₃.

Preferably, the moiety C₂-C₆-alkenyl is selected from the group consisting of CH═CH₂, CH═CHCH₃, CH₂CH═CH₂, CH═CHCH₂CH₃, CH₂CH═CHCH₃, and CH₂CH₂CH═CH₂.

Preferably, the moiety CO—C₁-C₂₀-alkyl is selected from the group consisting of COCH₃, COCH₂CH₃, COCH₂CH₂CH₃, COCH(CH₃)₂, COCH₂CH₂CH₂CH₃, COCH(CH₃)CH₂CH₃, COCH₂CH(CH₃)₂, COC(CH₃)₃, and COCH₂CH₂CH₂CH₂CH₂CH₃, CO-stearyl and palmityl

Preferably, the moiety CO₂—C₁-C₂-alkyl is selected from the group consisting of CO₂CH₃, CO₂CH₂CH₃, CO₂CH₂CH₂CH₃, CO₂CH(CH₃)₂, CO₂CH₂CH₂CH₂CH₃, CO₂CH(CH₃)CH₂CH₃, CO₂CH₂CH(CH₃)₂, CO₂C(CH)₃, and CO₂CH₂CH₂CH₂CH₂CH₂CH₃, CO₂-stearyl and palmityl

Preferably, the moiety OCO—C₁-C₂₀-alkyl is selected from the group consisting of OCOCH₃, OCOCH₂CH₃, OCOCH₂CH₂CH₃, OCOCH(CH₃)₂, OCOCH₂CH₂CH₂CH₃, OCOCH(CH₃)—CH₂CH₃, OCOCH₂CH(CH₃)₂, OCOC(CH₃)₃, and OCOCH₂CH₂CH₂CH₂CH₂CH₃, CO₂-stearyl and palmityl

Preferably, the moiety NH—C₁-C₆-alkyl is selected from the group consisting of NHCH₃, NHCH₂CH₃, NHCH₂CH₂CH₃, NHCH(CH₃)₂, NHCH₂CH₂CH₂CH₃, NHCH(CH₃)CH₂CH₃, NHCH₂CH(CH₃)₂, and NHC(CH₃)₃.

Preferably, the moiety CON(C₁-C₁₂-alkyl)₂ is selected from the group consisting of CON(CH₃)₂, CON(CH₂CH₃)₂, CON(CH₂CH₂CH₃)₂, CON(CH(CH₃)₂)₂, CON—(CH₂CH₂CH₂CH₃)₂, CON(CH(CH₃)CH₂CH₃)₂, CON(CH₂CH(CH₃)₂)₂, and CON(C(CH₃)₃)₂.

Preferably, the moiety SOₙ—C₁-C₆-alkyl is selected from the group consisting of SO₂CH₃, SO₂CH₂CH₃, SO₂CH₂CH₂CH₃, SO₂CH(CH₃)₂, SO₂CH₂CH₂CH₂CH₃, SO₂CH(CH₃)CH₂CH₃, SO₂CH₂CH(CH₃)₂, SO₂C(CH₃)₃, and SO₂CH₂CH₂CH₂CH₂CH₂CH₃.

Preferably, the moiety SOₙ—C₆-C₇-aryl is selected from the group consisting of SO₂-phenyl and SO₂—C₆H₄—CH₃.

Preferably, the moiety >CH—C₁-C₆-alkyl is selected from the group consisting of >CHCH₃, >CHCH₂CH₃, >CHCH₂CH₂CH₃, >CHCH(CH₃)₂, >CHCH₂CH₂CH₂CH₃, >CHCH(CH₃)CH₂CH₃, >CHCH₂CH(CH₃)₂, >CHC(CH₃)₃, and >CHCH₂CH₂CH₂CH₂—CH₂CH₃.

Preferably, the moiety C₁-C₆-alkylene-CONH₂ is selected from the group consisting of CH₂CONH₂, CH₂CH₂CONH₂, CH₂CH₂CH₂CONH₂, and CH₂CH₂CH₂CH₂CONH₂.

Preferably, the moiety C₁-C₆-alkylene-C₁-C₆-oxyalkyl is selected from the group consisting of CH₂OCH₂CH₃, CH₂OCH₂CH₂CH₃, CH₂CH₂OCH₃, CH₂CH₂OCH₂CH₃, CH₂CH₂OCH₂CH₂—CH₃, CH₂CH₂CH₂OCH₃, and CH₂CH₂CH₂OCH₂CH₃.

Preferably, the moiety C₁-C₆-alkylene-OCO—C₁-C₆-alkyl is selected from the group consisting of CH₂OCOCH₃, CH₂OCOCH₂CH₃, CH₂OCOCH₂CH₂CH₃, CH₂CH₂OCOCH₃, CH₂CH₂OCOCH₂CH₃ CH₂CH₂OCOCH₂CH₂CH₃, CH₂CH₂CH₂OCOCH₃, CH₂CH₂CH₂O—COCH₂CH₃, and CH₂CH₂CH₂OCOCH₂CH₂CH₃.

The pyrimidine derivatives according to the present invention may contain one or more chiral centers. When a single chiral center is present the compounds are preferably used in racemic form. However, in a preferred embodiment of the present invention the R or the S enantiomer of the pyrimidine derivatives is used in an enantiomeric excess ≧90% ee, more preferably ≧95% ee, most preferably ≧98% ee. The skilled person is aware of suitable methods of stereoselective synthesis or suitable methods of racemic resolution, such as HPLC on chiral solid phases, transient transformation into diastereomers (e.g. covalently bonded or via salt formation) and separation thereof, kinetic resolution by means of suitable enzymes, etc. When more than one chiral center is present in the pyrimidine derivatives, preferably one of the diastereomers is used in an diastereomeric excess ≧90% de, more preferably ≧95% de, most preferably ≧98% de.

Preferred compounds of general formula (IV-A) include:
2-piperazino-6-methyl-5-oxo-5,6-dihydro(7H)pyrro-[3,4-d]pyrimidine,
2-(4-methylpiperazino)-6-methyl-5-oxo-5,6-dihydro(7H)pyrro[3,4-d]pyrimidine,
2-(4-ethylpiperazino)-6-methyl-5-oxo-5,6-dihydro(7H)pyrro-[3,4d]pyrimidine,
2-piperidino-6-methyl-5-oxo-5,6-dihydro(7H)pyrro-[3,4d]-dipyrimidine,
2-(4-methylpiperidino)-6-methyl-5-oxo-5,6-dihydro(7H)pyrro-[3,4-d]pyrimidine,
2-(4-ethylpiperidino)-6-methyl-5-oxo-5,6-dihydro(7H)pyrro-[3,4-d]pyrimidine,
2-morpholino-6-methyl-5-oxo-5,6-dihydm(7H)pyrro-[3,4-d]pyrimidine,
2-thiomorpholino-6-methyl-5-oxo-5,6-dihydro(7H)pyrro-[3,4-d]pyrimidine,
2-piperazino-6-ethyl-5-oxo-5,6-dihydro(7H)pyrro-[3,4-d]pyrimidine,
2-piperazino-6-isopropyl-5-oxo-5,6-dihydro(7H)pyrro-[3,4-d]pyrimidine,
2-piperazino-6-n-butyl-5-oxo-5,6-dihydro(7H)pyrro-[3,4d]pyrimidine,
2-piperazino-6-sec-butyl-5-oxo-5,6-dihydro(7H)pyrro[3,4d]pyrimidine,
2-piperazino-6-tert-butyl-5-oxo-5,6-dihydro(7H)pyrro-[3,4-d]pyrimidine,
2-piperazino-4,6-dimethyl-5-oxo-5,6-dihydro(7H)pyrro-[3,4-d]pyrimidine,
2-piperazino-6,7-dimethyl-5-oxo-5,6-dihydro(7H)pyrro-[3,4-d]pyrimidine,
2-piperazino-6,7,7-trimethyl-5-oxo-5,6-dihydro(7H)pyrro-[3,4-d]pyrimidine,
2-piperidino-4,6-dimethyl-5-oxo-5,6-dihydro(7H)pyrro-[3,4-d]pyrimidine,
2-piperidino-6,7,7-trimethyl-5-oxo-5,6-dihydro(7H)pyrro-[3,4-d]pyrimidine,
2-piperazino-7-methyl-6-ethyl-5-oxo-5,6-dihydro(7H)pyrro-[3,4d]pyrimidine, and
2-piperazino-4-methyl-6-ethyl-5-oxo-5,6-dihydro(7H)pyrro-[3,4-d]pyrimidine.

Preferred compounds of general formula (IV-B) include:
2-piperazino-7-methyl-6-oxo-5,6-dihydro(7H)pyrro-[2,3-d]pyrimidine, 2-(4-methylpiperazino)-7-methyl-6-oxo-5,6-dihydro(7H)pyrro-[2,3-]pyrimidine,
2-(4-ethylpiperazino)-7-methyl-6-oxo-5,6-dihydro(7H)pyrro-[2,3-d]pyrimidine,
2-(4-N-acetylpiperzino)-7-methyl-6-oxo-5,6-dihydro(7H)pyrro-[2,3-d]pyrmidine,
2-(4-N-palmitoylpiperazino)-7-methyl-6-oxo-5,6-dihydro(7H)pyrro-[2,3-d]pyrimidine,
2-(4-N-stearoylpiperazino)-7-methyl-6-oxo-5,6-dihydro(7H)pyrro-[2,3-d]pyrimidine,
2-(4-N-phytanoylpiperazino)-7-methyl-6-oxo-5,6-dihydro(7H)pyrro-[2,3-d]pyrmidine,
2-piperidino-7-methyl-6-oxo-5,6-dihydro(7H)pyrro-[2,3-d]pyrimidine,
2-(4-methylpiperidino)-7-methyl-6-oxo-5,6-dihydro(7H)pyrro[2,3-d]pyrimidine,
2-(4-etylpiperidino)-7-methyl-6-oxo-5,6-dihydro(7H)pyrro-[2,3-d]pyrimidine,
2-morpholino-7-methyl-6-oxo-5,6-dihydro(7H)pyrro-[2,3-d]pyrimidine,
2-thiomorpholino-7-methyl-6-oxo-5,6-dihydro(7H)pyrro-[2,3-d]pyrimidine,
2-piperidino-7-ethyl-6-oxo-5,6-dihydro(7H)pyrro-[2,3-d]pyrimidine,
2-piperidino-7-n-propyl-6-oxo-5,6-dihydro(7H)pyrro-[2,3-d]pyrimidine,
2-piperidino-7-iso-propyl-6-oxo-5,6-dihydro(7H)pyrro-[2,3-d]pyrimidine,
2-piperidino-7-n-butyl-6-oxo-5,6-dihydro(7H)pyrro-[2,3-d]pyrimidine,
2-piperidino-7-tert-butyl-6-oxo-5,6-dihydro(7H)pyrro-[2,3-d]pyrimidine,
2-piperidino-5-methyl-6-oxo-5,6-dihydro(7H)pyrro-[2,3-d]pyrimidine,
2-piperazino-5-methyl-6-oxo-5,6-dihydro(7H)pyrro-[2,3-d]pyrimidine,
2-piperazino-4,7-dimethyl-6-oxo-5,6-dihydro(7H)pyrro-[2,3-d]pyrimidine,
2-piperidino-5,7-dimethyl-6-oxo-5,6-dihydro(7H)pyrro-[2,3-d]pyrimidine,
2-piperidino-5,5,7-trimethyl-6-oxo-5,6-dihydro(7H)pyrro-[2,3-d]pyrimidine,
2-piperazino-5,7-dimethyl-6-oxo-5,6-dihydro(7H)pyrro-[2,3-d]pyrimidine,
2-piperazino-5,7,7-trimethyl-6-oxo-5,6-dihydro(7H)pyrro-[2,3-d]pyrimidine,
2-piperidino-4-methyl-7-ethyl-6-oxo-5,6-dihydro(7H)pyrro-[2,3-d]pyrimidine, and
2-piperidino-5-methyl-7-ethyl-6-oxo-5,6-dihydro(7H)pyrro-[2,3d]pyrimidine.

Preferably, the pyrimidine derivatives are used according to the present invention for the preparation of a composition for providing a cosmetic effect being selected from the group consisting of prevention and/or treatment of wrinkles, prevention and/or treatment of acne, prevention and/or treatment of photo-damage, prevention and/or treatment of oxidative stress phenomena, prevention and/or treatment of cellulite, prevention and/or treatment of pigmentation disorders, thickening of the epidermis, promotion of hair growth, and inhibition and treatment of canities, rejuvenation of senescent skin cells. More preferably, the cosmetic effect is selected from the group consisting of prevention and/or treatment of wrinkles, thickening of the epidermis and promotion of hair growth and treatment of canities.

The present invention also relates to the use of the pyrimidine derivatives represented by general formula (I), (II) and (III-A-J), (IV-A) and (IV-B), respectively, or a pharmaceutically acceptable salt thereof for the preparation of a medicament for the treatment of a disorder selected from the group consisting of wrinkles, acne, photo-damage, oxidative stress phenomena, cellulite, pigmentation disorders, thinned epidermis, hair loss and senescence of skin cells.

Examples of cosmetically or pharmaceutically acceptable salts of the pyrimidine derivatives according to the present invention include salts formed from acids capable of forming cosmetically acceptable non-toxic and non-irritating acid-addition salts containing anions, such as the hydrochloride, hydrobromide, sulfate, bisulfite, phosphate, acid phosphate, acetate, palmitate, stearate, phytanate, maletate, fumarate, succinate, lactate, tartrate, benzoate, citrate, gluconate, glucanate, methanesulfonate, p-toluenesulfonate and naphthalenesulfonate, and their solvates, e.g. their hydrates, as well as the quaternary ammonium (or amine) salt and its solvate. Preferentially are salts with long chain carboxylic acids with more than 10 carbon atoms. Particular preferred for skin care emulsions is the stearate salt.

The pyrimidine derivatives according to the present invention are known in the art Regarding suitable methods for their preparation, refer to e.g. U.S. Pat. No. 4,959,368, EP-A 192 783 and EP-A 743 066 and the references cited therein.

In a preferred embodiment the present invention also relates to the use of the pyrimidine derivatives represented by general formulae (I), (II) and (III-A-J), (IV-A) and (IV-B) or a cosmetically acceptable salt thereof, respectively, for providing a cosmetic effect which is not a pharmaceutical effect. Preferably, said cosmetic effect is selected from the group consisting of prevention and/or treatment of wrinkles, prevention and/or treatment of acne, prevention and/or treatment of photo-damage, prevention and/or treatment of oxidative stress phenomena, prevention and/or treatment of cellulite, prevention and/or treatment of pigmentation disorders, thickening of the epidermis, promotion of hair growth, and inhibition of senescence of skin cells, more preferably consisting of prevention and/or treatment of wrinkles and canities, thickening of the epidermis and promotion of hair growth, prevention and treatment of skin cell recruitment disorders.

The present invention also relates to the use of the pyrimidine derivatives represented by general formulae (I), (II) and (III-A-J), (IV-A) and (IV-B) or non-toxic salts thereof for the prevention and/or treatment of wrinkles, prevention and/or treatment of acne, prevention and/or treatment of photo-damage, prevention and/or treatment of oxidative stress phenomena, prevention and/or treatment of cellulite, prevention and/or treatment of pigmentation disorders, thickening of the epidermis, protection against hair loss and promotion of hair growth, promotion of recolouring effect of canities and inhibition of senescence of skin cells.

The present invention also provides topical compositions in the form of creams, lotions, gels, tonics and oils comprising a pyrimidine derivative represented by general formula (I), (II) and (III-A-J), (IV-A) and (IV-B) or a cosmetically or pharmaceutically acceptable salt thereof, respectively, and a cosmetically or pharmaceutically acceptable excipient or diluent. The compositions are particularly useful for the topical treatment of unwounded skin or unwounded scalp. The creams, lotions, gels, tonics and oils are free of any live human cells or extracts thereof, and the creams, lotions, foams, gels, tonics and oils are applied topically to the skin and/or scalp preferably for the treatment of non-neurological diseases or conditions.

The present invention also provides cosmetic compositions comprising a pyrimidine derivative represented by general formula (I), (II), (III-AJ), (IV-A) and (IV-B) or a cosmetically acceptable salt thereof, respectively, and at least one additional active ingredient selected from anti-wrinkle/anti-atrophylanti-ageing actives, anti-oxidants/radical scavengers, flavonoids, anti-cellulite agents, tanning actives, skin lightening agents, sunscreen actives, particulate matter, hair growth actives, penetration enhancers/delivery agents, skin soothing actives, anti-acne actives, fragrances, dyes and pigments. Such compositions comprising a pyrimidine derivative and a further active ingredient are novel.

Anti-wrinkle/anti-atrophy/anti-ageing actives are disclosed e.g. in WO 2004/037213. Exemplary anti-wrinkle/anti-atrophy/anti-ageing actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, skin peel agents (e.g., phenol and the like), which enhance skin condition and Vitamin A and its derivatives.

Anti-oxidants/radical scavengers are known in the art. Especially preferred are antioxidants/radical scavangers chosen from the group consisting of amino acids (e.g. glycine, histidine, tyrosine, tryptophan) and their derivatives, imidazole (e.g. urocanic acid) and derivatives, peptides such as D,L-carnosine, D-carnosine, L-carnosine and derivatives (e.g. anserine), carotenoids, carotenes (e.g. α-carotene, β-rotene, lycopene) and derivatives, chlorogenic acid and derivatives, lipoic acid and derivatives (e.g. dihydrolipoic acid), aurothloglucose, propylthiouracil and other thiols (e.g. thioredoxine, glutathione, cysteine, cystine, cystamine and its glycosyt-, N-acetyl-, methyl-, ethyl-, propyl-, amyl-, butyl- and lauryl-, patmitoyl-; oleyl-, γ-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinsulfoximine, buthioninsulfone, penta-, hexa-, heptalhioninsulfoximine) in very low compatible doses (e.g. pmol to μmol/kg), (metal)-chelators (such as α-hydroxyfatty acids, palmic-, phytinic acid, lactoferrin), β-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyftetraisopalmitate, Mg-ascorbylphosphate, Na-ascorbylphosphate, ascorbylacetate), tocopherole and derivates (such as vitamin-E-acetate), mixtures of nat. vitamin E, vitamin A and derivatives (vitamin-A-palmitate and -acetate) as well as coniferylbenzoat, rutinic acid and derivatives, α-glycosylrutin, ferulic acid, furfurylidenglucitol, camosin, butylhydroxytoluene, butylhydroxyanisole, trihydroxybutyrophenone, urea and its derivatives, mannose and derivatives, zinc and derivatives (e.g. ZnO, ZnSO₄), selenium and derivatives (e.g. selenomethionin), stilbenes and derivatives (such as stilbenoxide, trans-stilbenoxide). Suitable derivatives of these active ingredients are e.g. salts, esters, ethers, sugars, nucleotides, nucleosides, peptides and lipids. One or more antioxidants/radical scavengers may be present in an amount about 0.01 wt. % to about 10 wt. % of the total weight of the composition of the present invention. Preferably, one or more antioxidants/radical scavengers are present in an amount about 0.1 wt. % to about 1 wt. %.

Suitable flavonoids are disclosed e.g. in WO 2004/037213 and in U.S. Pat. Nos. 5,686,082 and 5,688,367, all of which are herein incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. The term “substituted” as used herein means flavonoids wherein one or more hydrogen atoms of the flavonoid has been independently replaced with hydroxyl, C1-C8 alkyl, C1-C4 alkoxy, O-glycoside, and the like or a mixture of these substituents.

Anti-cellulite agents are known in the art and include, but are not limited to, xanthine compounds (e.g. caffeine, theophylline, theobromine and aminophylline).

Tanning actives are known in the art. If a tanning active is included with the compositions of the present invention, it is preferably present from approximately 0.1% to 20%. An example of a suitable tanning active is dihydroxyacetone.

Suitable skin lightening agents are disclosed e.g. in WO 2004/037213. When present, the compositions preferably contain from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in WO 95/34280 and WO 95/23780.

Suitable sunscreen actives are disclosed e.g. in WO 2004/037213. As used herein, “sunscreen active” includes both sunscreen agents and physical sunblocks. Suitable sunscreen actives may be organic or inorganic. Inorganic sunscreens useful herein include metallic oxides, in particular titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition. A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, α-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

Also particularly useful in the compositions are sunscreen actives such as those disclosed in U.S. Pat. No. 4,937,370 and U.S. Pat. No. 4,999,186. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Particularly preferred sunscreens are sunscreens on the basis of polysiloxanes such as Parsol SLX, described e.g. in EP 358 584, EP 538 431 and EP 709 080.

Suitable particulate matter is disclosed in WO 2004/037213. These particulates can be coated or uncoated, charged or uncharged. Charged particulate materials are disclosed in U.S. Pat. No. 5,997,887. Particulate materials useful herein include: bismuth oxychloride, iron oxide, mica, mica treated with barium sulfate and TiO₂, silica, nylon, polyethylene, talc, styrene, polypropylene, ethylene/acrylic acid copolymer, titanium dioxide iron oxide, bismuth oxychloride, sericite, aluminum oxide, silicone resin, barium sulfate, calcium carbonate, cellulose acetate, polymethyl methacrylate, and mixtures thereof.

Hair growth actives are known in the art. Hair growth actives or stimulants particularly suitable for use in compositions of the present invention may include but are not limited to benzalkonium chloride, benzethonium chloride, phenol, flavanoids, diphenhydramine hydrochloride, chlorophyllin derivatives, cholesterol, salicylic acid, cystine, methionine, red pepper tincture, benzyl nicotinate, di-menthol, peppermint oil, calcium pantothenate, panthenol, castor oil, hinokitiol, resorcinol, biotin, metabolic intermediates of the urea cycle (WO 94/09750), peptides, metal peptides and peptide derivatives.

Particularly preferred hair growth actives include: α-1,4 esterified disaccharides described in EP-A 0 064 012, esterfied oligosaccharides as described in EP-A 0 211 610, minoxidil glucuronides as described in EP 0 242 967, minoxidil sulphates as described in WO 86/04231, minoxidil, and other derivatives thereof as described in U.S. Pat. No. 4,139,619, ethylenediaminetetraacetic acid or salts thereof as described in U.S. Pat. No. 4,814,351, direct proteoglycanase inhibitors such as 1,10-phenanthroline as described in EP 0 277 428, glycosaminoglycanase inhibitors as described in EP 0 277 428 such as D-glucaro-1,4-lactone, glycosaminoglycanase inhibitors, as described in EP 0 277 428 such as N-acetylglucosamine, glycosaminoglycan chain cellular uptake inhibitors as described in EP 0 277 428 such as hexuronic acid and esters thereof, chemical inhibitors of glycosidase activity as described in EP 0 334 586 chosen from lactams, such as D-glucaro-1,5-lactam, chemical activators of protein kinase C enzymes as described in EP 0 334 585 chosen from diacylglycerols such as 1,2-dioleoyl-sn-glycerol, glycosaminoglycanase inhibitors as described in EP 0 348 184 such as 6-methyl-glucaro-1,4-lactone, glycosaminoglycanase inhibitors as described in EP 0 348 184 chosen from acylated monosaccharides such as 2-propionamido-2-deoxyglucose, esters of pyroglutamic acid as described in U.S. Pat. No. 4,774,255 such as pyroglutamic acid n-hexyl ester and pyroglutamic acid n-octyl ester, hexosaccharic acids or acylated hexosaccharic acids, or salts or esters thereof, as described in EP 0 378 388 such as glucosaccharic acid, and its disodium salt, aryl-substituted ethylenes as described in EP 0 403 238 such as 1,1-dicarboxy-2-(4-hydroxyphenyl)ethylene.

Penetration enhancers/delivery agents are compounds which improve the delivery of the pyrimidine derivative or salt thereof to its site of action or target in the hair follicle, hair shaft, epidermal stem cell compartment, or the like. Lecithin or non-lecithin liposome or other forms of particulated capsules may be employed to enable delivery. Examples of penetration enhancers/delivery agents may include fatty acids and their salts, in particular stearic and plamitic acid, 1,2-propane diol, 1,2-hexan diol and 1,2-pentan diol

Examples of penetration enhancers/delivery agents include in particular 2-methyl propan-2-ol, propan-2-ol, ethyl-2-hydroxypropanoate, hexan-2,5-diol, POE(2) ethyl ether, di(2-hydroxypropyl)ether, pentan-2,4-diol, acetone, POE(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, POE ester of oleyl alcohol, oleyl alcohol, lauryl alcohol, dioctyl adipate, dicapryl adipate, diisopropyl adipate, diisopropyl sebacate, dibutyl sebacate, diethyl sebacate, dimethyl sebacate, dioctyl sebacate, dibutyl suberate, dioctyl azelyate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, and 1-dodecylazacycloheptan-2-one.

Skin soothing actives include panthenoic acid derivatives (inciuding panthenol, dexpanthenol, ethyl panthenol), aloe vera, allantoin, bisabolol and dipotassium glycyrrhizinate. Anti-acne actives include Vitamin C and Its derivatives.

If nothing else is stated, the additional active ingredient is present in the compositions of the present invention in an amount of preferably 0.1 to 20, more preferably of 1 to 10 wt.-%, based on the weight of the composition.

In a preferred embodiment the composition according to the present invention is a cosmetic preparation comprising a pyrimidine derivative represented by general formula (I), (II), (III-A-J), (IV-A) and (IV-B) or a cosmetically acceptable salt thereof, respectively, and a cosmetically acceptable excipient or diluent. In another preferred embodiment the composition according to the present invention is a pharmaceutical preparation comprising a pyrimidine derivative represented by general formula (I), (II), (III-A-J), (IV-A) and (IV-B) or a pharmaceutically acceptable salt thereof, respectively, and a pharmaceutically acceptable excipient or diluent.

The compositions of the present invention include cosmetic preparations and pharmaceutical preparations. These preparations are preferably topical preparations, but the compositions according to the present invention may also be administered by injection (e.g. intravenously, intramuscularly, subcutaneously, and the like).

The term “cosmetic preparation” or “cosmetic composition” as used in the present specification refers to cosmetic compositions as defined under the heading “Kosmetika” in Römpp Lexikon Chemie, 10th edition 1997, Georg Thieme Verlag Stuttgart, N.Y.

The cosmetic preparations of the present invention contain the pyrimidine derivative together with cosmetically acceptable excipients or diluents. If nothing else is stated, the excipients, additives, diluents, etc. mentioned in the following are suitable for both pharmaceutical and cosmetic preparations.

If nothing else is stated, in this specification parts and percentages are per weight and are based on the weight of the composition.

Preferably, the compositions of the present invention are topical preparations, such as liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, ointments, plasters, suspensions, powders, creams, cleanser, soaps and other usual compositions, which can also be applied by pens, as masks or as sprays.

More preferably, the compositions of the present invention are topical preparations selected from the group consisting of liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, creams, cleanser and soaps.

The compositions of the present invention usually contain cosmetic or pharmaceutical adjuvants and additives, such as fatty substances/oils, water, organic solvents, silicones, thickeners, softeners, emulsifiers, antifoaming agents, moisturizers, surfactants, filters, sequestering agents, anionic, cationic, nonionic or amphoteric polymers or mixtures thereof, propellants, acidifying or basifying agents, or any other ingredients usually formulated into cosmetics or medicaments.

Typically topical preparations also contain surface active ingredients like emulsifiers, solubilizers and the like. An emulsifier enables two or more immiscible components to be combined homogeneously. Moreover, the emulsifier acts to stabilize the composition. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP elcosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer and mixtures thereof. The preferred emulsifiers are cetyl phosphate (Amphisol® A), diethanolamine cetyl phosphate (Amphisol®), potassium cetyl phosphate (Amphisol® K), PVP Elcosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount about 0.01 wt. % to about 20 wt. % of the total weight of the composition of the present invention. Preferably, about 0.1 wt. % to about 10 wt. % of emulsifiers are used.

The lipid phase of the topical preparations can advantageously be chosen from:
mineral oils and mineral waxes;
oils such as triglycerides of caprinic acid or caprylic acid, preferable castor oil;
oils or waxes and other natural or synthetic oils, in an preferred embodiment esters of fatty acids with alcohols e.g. isopropanol, propylene glycol, glycerin or esters of fatty alcohols with carbonic acids or fatty acids;
alkylbenzoates; and/or
silicone oils such as dimethylpolysiloxane, diethylpolysiloxane, diphenylpolysiloxane, cyclomethicones
   and mixtures thereof. Preferably, however, the lipid phase does not contain a mineral oil.

Exemplary fatty substances which can be incorporated in the oil phase of the emulsion, microemulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropyl-myristate, isopropylpalmitate, isopropylstearate, isopropyloleate, n-butylstearate, n-hexyllaureate, n-decyloleat, isooctylstearate, isononylstearate, isononylisononanoate, 2-ethyl hexylpaImitate, 2-ethylhexyllaurate, 2-hexyldecylstearate, 2-octyidodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the topical preparations of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyidimethicone, hexamethylcyclo-trisiloxane, polydimethylsiloxane, poly(methylphenylsiloxane) and mixtures thereof.

Other fatty components which can advantageously be incorporated in topical preparations of the present invention are isoeikosane; neopentylglycoldlheptanoate; propyleneglycol-dicaprylatel dicaprate; caprylicl capric/diglycerylsuccinate; butyleneglycol caprylat/caprat; C₁₂₋₁₃-alkyllactate; di-C₁₂₋₁₃ alkyltartrate; triisostearin; dipentaerythrityl hexacaprylaV hexacaprate; propyleneglycolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecyllsononanoate.

The oily phase of the compositions of the present invention can also contain natural vegetable or animal waxes such as bee wax, china wax, bumblebee wax and other waxes of insects as well as shea butter and cocoa butter.

A moisturizing agent may be incorporated into a topical preparation of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, caprictcaprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₁₋₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 20 wt. % of the total weight of the composition. The preferred amount of emollient is about 2 wt. % to about 15 wt. %, and most preferably about 4 wt. % to about 10 wt. %.

Moisturizers that bind water, thereby retaining it on the skin surface are called humectants. Suitable humectants can be incorporated into a topical preparation of the present invention such as glycerin, polypropylene glycol, polyethylene glycol, lactic acid, pyrrolidone carboxylic acid, urea, phospholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel® 1000 (CAS-Nr. 178463-23-5) by SOLABIA S. One or more humectants are optionally present at about 0.5 wt. % to about 8 wt. % in a composition of the present invention, preferably about 1 wt. % to about 5 wt. %.

The aqueous phase of the preferred topical preparations of the present invention can contain the usual cosmetic or pharmaceutical additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, foam stabilizers; electrolytes and especially one or more thickeners. Thickeners that may be used in formulations of the present invention to assist in making the consistency of a product suitable include carbomer, siliciumdioxide, magnesium and/or aluminum silicates, beeswax, stearic acid, stearyl alcohol-polysaccharides and their derivatives such as xanthan gum, hydroxypropyl cellulose, polyacrylamides, acrylate crosspolymers preferably a carbomer, such as Carbopole® of type 980, 981, 1382, 2984, 5984 alone or mixtures thereof. Suitable neutralizing agents which may be included in the composition of the present invention to neutralize components such as e.g. an emulsifier or a foam builderlstabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the composition of the present invention, preferably, 1 wt. % to about 5 wt. %.

The addition of electrolytes into the composition of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the composition of the present invention.

The topical preparations of the present invention can preferably be provided in the form of a lotion, a thickened lotion, a gel, a cream, a milk, an ointment, a powder or a solid tube stick and can be optionally be packaged as an aerosol and can be provided in the form of a mousse, foam or a spray. The compositions according to the invention can also be in the form of a suspension or dispersion in solvents or fatty substances, or alternatively in the form of an emulsion or microemulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactants.

The topical application is preferably at least once per day, e.g. two or three times a day. Usually it takes at least two weeks until the desired effect starts to show up and some more weeks until the maximum benefit is achieved. However, it can also take several weeks or even months until the desired effect is achieved. In some cases it might be advisable to perform the treatment in intervals to maximize the benefit.

The amount of the topical preparation which is to be applied to the skin depends on the concentration of the active ingredients in the compositions and the desired cosmetic or pharmaceutical effect. For example, application can be such that a cream is applied to the skin. A cream is usually applied in an amount of 2 mg cream/cm² skin. The amount of the composition which is applied to the skin is, however, not critical, and if with a certain amount of applied composition the desired effect cannot be achieved, a higher concentration of the active ingredients can be used e.g. by applying more of the composition or by applying compositions which contain more active ingredient.

According to the invention for preparing the compositions the active ingredients can be used as such or in an encapsulated form, for example in a liposomal form. Liposomes are preferably formed with lecithins with or without addition of sterols or phytosterols. The encapsulation of the active ingredients can be alone or together with other active ingredients.

A preferred embodiment is the use of hydrophobic counter anions, including but not limited to palmitate or stearate for better delivery of the active materials to the desired target place. Alternatively there can also materials be used to permeate temporarily the barrier including glycols, particular 1,2-propylene glycol, 1,2-pentylenglycol, Ethanol or DMSO.

In the composition of the invention the pyrimidine derivative is contained in an amount of preferably 0.0001 wt.-% to about 50 wt.-%, based on the total weight of the composition. More preferably, the pyrimidine derivative is contained in the composition in an amount of about 0.01 wt.-% to about 20 wt.-%, more preferably in an amount of about 0.01 wt.-% to about 1 wt.-%, in particular in an amount of about 0.1 wt.-%, based on the total amount of the composition.

Regarding the kind of the topical preparation and the manufacture of the topical preparations as well as for further suitable additives, it can be referred to the pertinent literature, e.g. to Novak G. A., Die kosmetischen Präparate—Band 2, Die kosmetischen Präparate—Rezeptur, Rohstoffe, wissenschaftliche Grundlagen (Verlag für Chem. Industrie H. Ziolkowski K G, Augsburg).

The compositions of the present invention can also be in the form of injectable preparations, in particular if the compositions are for promoting hair growth. The manufacture of injectable preparations is known to a skilled person and one can consult the pertinent literature in particular Remington, as cited above. Injection would also include microinjection designed to generate just one hair per prick. Preferable formulations for this purpose are sterile water-based salt solution of the compounds.

The pyrimidine derivatives or the cosmetically or pharmaceutically acceptable salts thereof can also be present as hydrates or solvates, and the hydrates and solvates of the active ingredients are also encompassed by the present invention.

The following examples further illustrate the invention, but they should not be construed as limiting the invention.

### EXAMPLE 1-2 HAIR LOSS SCALP TREATMENT SERA

| Formulation No | 1 | 2 |
|---|---|---|
| Ingredients | % (w/w) | % (w/w) |
| Water | Ad. 100 | Ad. 100 |
| Ethanol | 8.00 | 8.00 |
| Isopropanol | 4.00 | 4.00 |
| Propylene Glycol | 5.00 | 5.00 |
| D-Panthenol | 0.20 | 0.20 |
| PEG-12 Dimethicone | 0.20 | 0.20 |
| PEG-40 Hydrogenated Castor Oil | 4.00 | 4.00 |
| Phytantriol | 0.05 | 0.05 |
| Vitamin E Acetate | 0.10 | 0.10 |
| 2-Piperazino-7-methyl-6-oxo-5,6- | 0.1 | |
| dihydro(7H)pyrro-[2,3-d]pyrimidine | | |
| 2-(4-N-Acetylpiperazino)-7-methyl-6-oxo-5,6- | | 0.1 |
| dihydro(7H)pyrro-[2,3-d]pyrimidine, | | |
| Citric acid 10% | q.s. | q.s. |

Mix ingredients and adjust pH to 6 with Citric acid.

### EXAMPLES 3-4 FACIAL ANTI-WRINKLE TREATMENT FORMULATIONS

| Formulation No. | 3 | 4 |
|---|---|---|
| Ingredients | % (w/w) | % (w/w) |
| Glyceryl Myristate | 5.00 | 5.00 |
| Cetyl Alcohol | 2.00 | 2.00 |
| Cetyl Phosphate | 2.00 | 2.00 |
| Isopropyl Myristate | 10.00 | 10.00 |
| Tocopheryl Acetate | 0.30 | 0.30 |
| Almond Oil | 2.00 | 2.00 |
| Phenoxyethanol & Methylparaben & | 0.60 | 0.60 |
| Ethylparaben & Propylparaben & | | |
| Butylparaben & Isopropylparaben | | |
| Sodium hydroxide 10% | q.s. | q.s. |
| Water | Ad. 100 | Ad. 100 |
| D-Panthenol | 0.20 | 0.20 |
| Disodium EDTA | 0.10 | 0.10 |
| Propylene Glycol | 4.00 | 4.00 |
| Polyacrylamide & C13-14 Isoparaffin & | 2.00 | 2.00 |
| Laureth-7 | | |
| 2-Piperazino-7-methyl-6-oxo-5,6- | 0.20 | — |
| dihydro(7H)pyrro-[2,3-d]pyrimidine | | |
| 2-(4-N-Acetylpiperazino)-7-methyl-6-oxo-5,6- | — | 0.2 |
| dihydro(7H)pyrro-[2,3-d]pyrimidine | | |
| Myristic acid | 0.1 | |

### Hair Growth Effects

The protection and stimulation effects of the compounds of this invention can be assessed by in vitro models for example reported in: “Human hair growth in vitro: a model for the study of hair follicle biology”, Philpott M P, Anders D, Westgate G E, Kealey T, J Dermatol Sci 1994 July; 7 Suppl:S55-72 or “Enhanced in vitro hair growth at the air-liquid interface: minoxidil preserves the root sheath in cultured whisker follicles”, Waldon D J; Kawabe Ur; Baker C A; Johnson G A; Buhl A E, In Vitro Cell Dev Biol Anim 1993 July; 29A (7): 555-61.

The influence of the compounds of this invention on human hair follicle length and cell proliferation can be assessed in vitro for example using a hair organ culture model in which hair follicles are excised from the scalp and grown and measured under controlled laboratory conditions, as described in “Influence of melatonin and melatonin antagonists on hair shaft length and proliferation in cultivated human hair follicles” T. W. Fisher et al., Abstract EHRS conference Marburg, 2000.

Stimulation of hair growth or hair growth protection can be investigated with a mouse model described for example in WO 98/7273. Instead of using Cyclophosphamide (Neostar, Pharmacia) to damage hair follicles, Mitomycin or Methotrexate can be used. However it is also possible to detect hair growth acceleration with newborn mice. They have a synchronized hair cycle and after approximately 3 weeks all hair follicles go into the telogen phase. Then the animals are treated and it is evaluated how fast and to what extent the hair is growing. Similar tests in vitro and in vivo can also be found in J. Invest. Dermato. symposium proceedings 3rd Int. Meeting of Hair Research Societies, 8/1, p. 39-45 (2003),

It also is possible to perform a clinical study involving males suffering from alopecia using the TrichoScan analysis tool described in R. Hoffmann, J. Invest. Dermato. symposium proceedings 3rd int. Meeting of Hair Research Societies, 811, p. 109-115 (2003).

1. Use of a pyrimidine derivative represented by general formula (I) 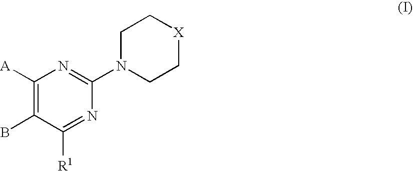
wherein
R¹ is H, C₁-C₁₂-alkyl, C₁-C₆-oxyalkyl, C₁-C₆-thioalkyl, C₁-C₆-alkylene-C₁-C₆-oxyalkyl, C₁-C₆-alkylene-CONH₂, CO—C₁-C₆-alkyl, or C₁-C₆-alkylene-OCO—C₁-C₆-alkyl;
X is —O—, —S—, —CH₂—, >CH—C₁-C₆-alkyl or >NR² wherein R² is H, OH, C₁-C₆-alkyl, C₂-C₆-alkenyl, phenyl, benzyl, CH(phenyl)₂, CO—C₁-C₂₀-alkyl, CO₂—C₁-C₂₀-alkyl, or SOₙ—C₁-C₂₀-alkyl or SOₙ—C₂₀-C₇-aryl (wherein index n is an integer of 0 to 2);
A is H, NH₂, NH—C₁-C₆-alkyl, C₁-C₆-oxyalkyl, or CO₂—C₁-C₆-alkyl;
B is H, CO₂—C₁-C₆-alkyl, CON(C₁-C₁₂-alkyl)₂, C₁-C₆-oxyalkyl, or CH₂CH₂OH; or
A and B together with the carbon atoms to which they are attached form a 5- to 7-membered carbocyclic ring or a heterocyclic ring having N, O or S as the hetero atom;
or a cosmetically acceptable salt thereof;
for the preparation of a composition for providing a cosmetic effect.

2. Use according to claim 1, characterized in that the pyrimidine derivative is represented by general formula (II) 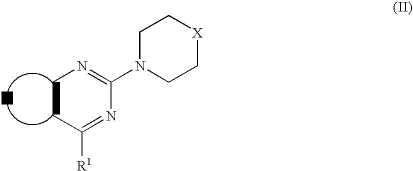
wherein R¹ and X are defined as in claim 1 and wherein
Y is a moiety selected from the group consisting of the moieties represented by general formulae (III-A) to (III-J) 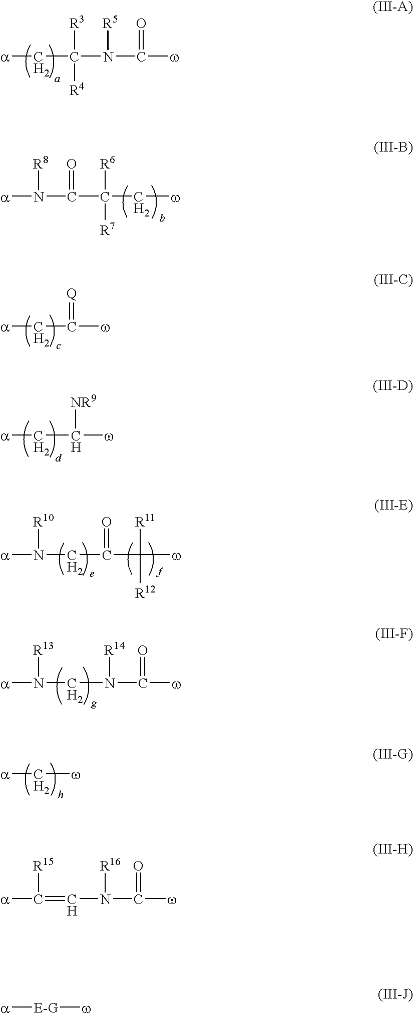
wherein
α and ω are the positions to be bound to positions 4 and 5 of the pyrimidine ring in general formula (II);
indices a and b each are independently an integer of 0 to 2;
indices c and d each are independently an integer of 2 to 4;
indices e and f each are independently an integer of 0 to 2, provided that
1≦(e+f)≦3;
index g is 1 or 2;
index h is 3 or 4;
Q is ═O or ═NR² (wherein R² is independently defined as in claim 1);
E-G is —OCH₂CH₂—, —OC(CH₃)═CH—, —CH₂OCO—, —OCOCH₂—, —CH₂CH(CH₃)OCO—, —N(CH₃)CH₂CH₂—, —CH═CH—CH═CH—, —CH═C(OCH₃)═CH—, or 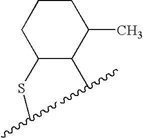
R³, R⁴, R⁶ and R⁷ each are independently H, C₁-C₁₂-alkyl, C₁-C₆-oxyalkyl, C₁-C₆-alkylene-C₁-C₆-oxyalkyl, C₁-C₆-alkylene-CONH₂, CO—C₁-C₆-alkyl, or C₁-C₆-alkylene-OCO—C₁-C₆-alkyl; and
R⁵, R⁸, R⁹, R¹⁰, R¹¹, R¹², R¹³, R¹⁴, R¹⁵ and R¹⁶ each are independently H or C₁-C₆-alkyl;
or a cosmetically acceptable salt thereof.

3. Use according to claim 2, characterized in that position a in general formulae (III-A) to (III-F) is bound to position 4 of the pyrimidine ring in general formula (II) and position ω in general formulae (III-A) to (III-F) is bound to position 5 of the pyrimidine ring in general formula (II).

4. Use according to, claim 1 characterized in that the pyrimidine derivative is a compound represented by general formula (IV-A) or (IV-B) 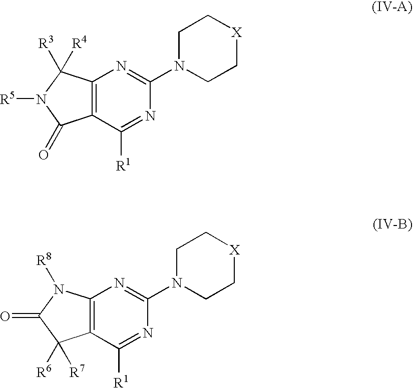
wherein X, R¹, R³, R⁴, R⁵, R⁶, R⁷ and R⁸ are defined as in claim 2.

5. Use according to claim 1, characterized in that the cosmetic effect is selected from the group consisting of prevention and/or treatment of wrinkles, prevention and/or treatment of acne, prevention and/or treatment of photo-damage, prevention and/or treatment of oxidative stress phenomena, prevention and/or treatment of cellulite, prevention and/or treatment of pigmentation disorders, promotion of thickening of the epidermis, promotion of hair growth, prevention and/or treatment of canities, promotion of re-pigmentation of canities, prevention and/or treatment of hair thinning, promotion of thickening of vellus hairs and inhibition of senescence of skin cells.

6. Use according to claim 5, characterized in that the cosmetic effect is selected from the group consisting of prevention and/or treatment of wrinkles, thickening of the epidermis, promotion of hair growth and prevention and/or treatment of greying hair.

7. Use of a pyrimidine derivative as defined in claim 1 or a pharmaceutically acceptable salt thereof for the preparation of a pharmaceutical composition for the treatment of a disorder selected from the group consisting of wrinkles, acne, photo-damage, oxidative stress phenomena, cellulite, pigmentation disorders, thinned epidermis, hair loss, disturbances in hair follicle formation or cycling, hair thinning, canities formation and senescence of skin cells.

8. Use according to claim 1, wherein the composition is a topical composition.

9. Use according to claim 1, wherein the composition is an injectable composition.

10. Composition comprising a pyrimidine derivative as defined in claim 1 or a cosmetically or pharmaceutically acceptable salt thereof and a cosmetically or pharmaceutically acceptable excipient or diluent in combination with at least one active ingredient selected from anti-wrinkle/anti-atrophy/anti-ageing actives, anti-oxidants/radical scavengers, flavonoids, anti-cellulite agents, tanning actives, skin lightening agents, sunscreen actives, particulate matter, hair growth actives, penetration enhancers/delivery agents, skin soothing actives, anti-acne actives, fragrances, dyes and pigments.

11. Composition according to claim 10, characterized in that it is a topical composition.

12. Composition according to claim 10, characterized in that it is a cosmetic composition.

13. Composition according to claim 10, characterized in that it is a pharmaceutical composition.

14. Composition according to claim 10, characterized in that it is a preparation selected from the group consisting of liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, creams, cleanser and soaps.

15. Composition according to claim 10, characterized in that it contains the pyrimidine derivative in a concentration of 0.001 to 50 wt.-%, based on the total weight of the composition.

16. Composition according to claim 15, characterized in that it contains the pyrimidine derivative in a concentration of 0.01 to 1 wt.-%, based on the total weight of the composition.
